(19) United States
(12) Patent Application Publication
(10) Pub. No.: US 2026/0160710 A1
(43) Pub. Date: Jun. 11, 2026

(54) METHOD AND SYSTEM FOR MEASURING SOIL BULK DENSITY OF TILLAGE LAYER BASED ON MULTI-CHANNEL GROUND PENETRATING RADAR

(71) Applicant: Institute of Soil Science, CAS, Nanjing City (CN)

(72) Inventors: Xicai Pan, Nanjing City (CN); Cong Tang, Nanjing City (CN); Jiabao Zhang, Nanjing City (CN); Jienan Xu, Nanjing City (CN); Lei Qiao, Nanjing City (CN)

(21) Appl. No.: 19/410,638

(22) Filed: Dec. 5, 2025

(30) Foreign Application Priority Data
Dec. 5, 2024 (CN) 202411775403.3

(51) Int. Cl. G01N 22/04; G01N 33/24; G01S 7/02; G01S 7/40; G06N 3/0464; G06N 3/08

(52) U.S. Cl. CPC G01N 22/04; G01N 33/245; G01N 33/246; G01S 7/0232; G01S 7/4021; G06N 3/0464; G06N 3/08

(57) ABSTRACT

A method and system for measuring soil bulk density in a farmland tillage layer based on a multi-channel ground penetrating radar are provided, relating to the field of soil bulk density measurement. The multi-channel ground penetrating radar can be configured to directly measure large area of soil and to acquire a corresponding reciprocal of an average amplitude envelope of a tillage layer electromagnetic wave signal acquired by antennas with different spacings, and a volumetric water content is predicted by a model based on the reciprocal of the average amplitude envelope; then the volumetric water content and the reciprocal of the average amplitude envelope are used as an input of a trained bulk density inversion machine learning model.

### CROSS-REFERENCE TO RELATED APPLICATION

This patent application claims the benefit and priority of Chinese Patent Application No. 202411775403.3 filed with the China National Intellectual Property Administration on Dec. 5, 2024, the disclosure of which is incorporated by reference herein in its entirety as part of the present application.

### TECHNICAL FIELD

The present disclosure relates to the field of soil bulk density measurement, and in particular to a method and system for measuring soil bulk density of a farmland tillage layer based on a multi-channel ground penetrating radar.

### BACKGROUND

Farmland soil is divided into a tillage layer, a plow pan, a subsoil layer, and a substratum from top to bottom, among which the tillage layer plays a crucial role in agricultural production due to its rapid soil material transformation, excellent permeability, and high nutrient content, and has functions mainly reflected in providing nutrients required for plant growth, ensuring excellent physical properties, regulating soil temperature, enhancing the buffering capacity of physical and chemical properties, and the like. Soil bulk density means the mass of soil solids per unit volume of undisturbed soil and is a metric for measuring soil compaction degree. The magnitude of the soil bulk density is a comprehensive reflection of soil physical properties such as texture, structure, and porosity. An appropriate soil bulk density in the tillage layer can retain moisture and nutrients better, thereby creating a stable and supportive environment for crop growth. In agricultural production. Unreasonable tillage practices, fertilization management, and irrigation management may increase the soil bulk density of the tillage layer, leading to enhanced soil compaction, and diminished water and air permeability. This is not conducive to root penetration and crop growth, and may cause loss of water and nutrients. Therefore, accurate measurement of the soil bulk density in the farmland tillage layer is crucial for assessing soil physical properties, fertility status, and formulating agricultural management strategies.

Multiple point-scale-based detection techniques exist for measuring soil bulk density in the tillage layer. Among them, the core cutter method is the most established, offering the advantages such as simple device, ease of operation, and accurate results. A number of alternative techniques for determining soil bulk density based on a relationship between mass and volume include a paraffin wax method, a mercury displacement method, and a sand-filling method. Furthermore, several methods enable the indirect determination of soil bulk density by measuring other physical properties of the soil, such as a ray method and an electromagnetic probe measurement method. Despite their ability to provide relatively accurate bulk density detection data at a point scale, these methods are inefficient and costly, thus impractical for large-area, rapid, and continuous detection. Many of these methods are ill-suited for the loose tillage layer soil.

### SUMMARY

For the foregoing limitations in the prior art, the present disclosure provides a method and system for measuring soil bulk density of a farmland tillage layer based on a multi-channel ground penetrating radar, which solve the problem that the existing soil bulk density detection method is difficult to achieve large-area, rapid and continuous detection and unsuitable for detecting the loose tillage layer soil.

To achieve the foregoing objective, the technical solution employed by the present disclosure as follows.

A method for measuring soil bulk density of a farmland tillage layer based on a multi-channel ground penetrating radar is provided, including following steps:
step S1: constructing a relationship model between a reciprocal of an average amplitude envelope and a volumetric soil water content in a farmland tillage layer;
step S2: constructing a trained bulk density inversion machine learning model;
step S3: obtaining an initial detection signal by detecting a target farmland tillage layer through a ground penetrating radar;
step S4: preprocessing the initial detection signal, and extracting a reciprocal of an average amplitude envelope of preprocessed initial detection signal within a time interval from time zero to a moment when an amplitude envelope reaches its maximum;
step S5: obtaining a corresponding predicted volumetric water content according to the reciprocal of the average amplitude envelope obtained in step S4 and the relationship model between the reciprocal of the average amplitude envelope and the volumetric soil water content in the farmland tillage layer; and
step S6: serving the reciprocal of the average amplitude envelope of a tillage layer electromagnetic wave signal acquired by antennas with different spacings obtained in step S4 and the predicted volumetric water content obtained in step S5 as inputs of the trained bulk density inversion machine learning model, and predicting soil bulk density of the farmland tillage layer through the trained bulk density inversion machine learning model to obtain a soil bulk density measurement result of the farmland tillage layer.

Further, a specific method for constructing the relationship model between the reciprocal of the average amplitude envelope and the volumetric soil water content in the farmland tillage layer through testing in step S1 includes following sub-steps:
step S1-1: selecting soil from the target farmland tillage layer and subjecting selected soil to air-drying and sieving treatment;
step S1-2: based on the soil treated in step S1-1, constructing a plurality of simulated samples with different bulk densities and volumetric water content conditions in a wooden box, where a soil thickness in the wooden box is greater than or equal to 20 cm;
step S1-3: placing the ground penetrating radar on a surface of each simulated sample constructed in step S1-2 with more than three antenna spacings to collect an initial detection signal corresponding to each simulated sample;
step S1-4: preprocessing the initial detection signal corresponding to each simulated sample collected in step S1-3, and extracting a reciprocal of an average amplitude envelope of a preprocessed detection signal corresponding to each simulated sample within a time interval from time zero to a moment when an amplitude envelope reaches its maximum;
step S1-5: sampling each simulated sample using a core cutter method and drying each simulated sample, and calculating an actual volumetric water content and bulk density of each simulated sample; and
step S1-6: training a shallow neural network model by using the reciprocal of the average amplitude envelope obtained in step S1-4 as a training sample of the shallow neural network model and the actual volumetric water content of each simulated sample as a label of the training sample of the shallow neural network model to obtain a trained shallow neural network model, that is, obtaining the relationship model between the reciprocal of the average amplitude envelope and the volumetric soil water content in the farmland tillage layer.

Further, in a process of training the shallow neural network model, stability of the shallow neural network model is evaluated through cross-validation, a prediction accuracy of the shallow neural network model is evaluated through a root mean square error metric, and a training of the shallow neural network model is finished when the stability and prediction accuracy of the shallow neural network model meet a first predetermined condition.

Further, specific method for constructing the trained bulk density inversion machine learning model in step S2 includes:
training a convolutional neural network (CNN) model with a corresponding reciprocal of an average amplitude envelope and the actual volumetric water content of the simulated sample as a training sample and the actual bulk density of the simulated sample as a label to obtain a trained convolutional neural network model, that is, obtaining the trained bulk density inversion machine learning model.

Further, in a process of training the convolutional neural network model, stability of the convolutional neural network model is evaluated through cross-validation, a prediction accuracy of the convolutional neural network model is evaluated through a root mean square error metric, and a training of the convolutional neural network model is completed when the stability and prediction accuracy of the convolutional neural network model meet a second predetermined condition.

Further, the specific method for preprocessing the initial detection signal includes following steps:
performing time-zero calibration on the initial detection signal by using timing of Wide Angle Reflection and Refraction (WARR) signals measured at different spacings in air, to obtain a time-zero calibrated signal;
eliminating a low-frequency noise from the time-zero calibrated signal using a De-Wow filter, to obtain a low-frequency filtered signal; and
for the low-frequency filtered signal, comparing the amplitude energy differences among multiple sets of fixed-spacing airwave from N measurements, setting an amplitude energy of fixed-spacing airwave from one selected measurement as a reference, and correcting amplitude energies of fixed-spacing airwaves from remaining N−1 measurements to a same level, to obtain an amplitude corrected signal, that is, obtaining the preprocessed initial detection signal.

A system for implementing the method for measuring soil bulk density of a farmland tillage layer based on a multi-channel ground penetrating radar is provided, including a data collection module, a multi-channel ground penetrating radar measurement module, a data transmission module, and an upper computer.

The data collection module includes a camera and a Real-Time Kinematic (RTK) positioning device, where the camera is configured to collect a surface image of a target farmland tillage layer, and the RTK positioning device is configured to monitor position information of a point detected by the ground penetrating radar in real time by means of RTK positioning technology.

The multi-channel ground penetrating radar measurement module is configured to detect the target farmland tillage layer to acquire an initial detection signal.

The data transmission module is configured to transmit data obtained by both the data collection module and the multi-channel ground penetrating radar measurement module to the upper computer through a wireless communication network.

The upper computer includes a feature extraction module and a data integration intelligent processing module, where the feature extraction module is configured to preprocess the initial detection signal and extract a reciprocal of an average amplitude envelope of a preprocessed initial detection signal within a time interval from time zero to a moment when the amplitude envelope reaches its maximum.

The data integration intelligent processing module is configured to: construct a relationship model between the reciprocal of the average amplitude envelope and a volumetric soil water content in a farmland tillage layer; construct a trained bulk density inversion machine learning model; acquire a corresponding predicted volumetric water content according to the relationship model between the reciprocal of the average amplitude envelope and the volumetric soil water content in the farmland tillage layer; predict, based on the reciprocal of the average amplitude envelope and the predicted volumetric soil water content, soil bulk density of the farmland tillage layer through the trained bulk density inversion machine learning model to obtain a soil bulk density measurement result of the farmland tillage layer, and to store and display data collected by the data collection module with a corresponding soil bulk density measurement result of the farmland tillage layer.

The present disclosure has beneficial effects as follows.

1. According to the present disclosure, the ground penetrating radar can be configured to directly measure large area of soil and to acquire a corresponding reciprocal of an average amplitude envelope, and a volumetric water content is predicted by a model based on the reciprocal of the average amplitude envelope; then the volumetric water content and the reciprocal of the average amplitude envelope are used as inputs of a trained bulk density inversion machine learning model. The scheme can achieve high-resolution soil bulk density measurement and is suitable with loose tillage layer soil, so that the measurement accuracy and efficiency are effectively improved.

2. According to the present disclosure, a relationship model between the reciprocal of the average amplitude envelope and the volumetric soil water content in the farmland tillage layer is constructed by using the soil in the target farmland tillage layer. By incorporating the volumetric soil water content and soil bulk density of the target farmland tillage layer measured during actual measurement tests, a trained machine learning model for bulk density inversion can be constructed for the soil in the target farmland tillage layer, so that a more accurate prediction value can be obtained when the constructed relationship model between the reciprocal of the average amplitude envelope and the volumetric soil water content in the farmland tillage layer and the trained bulk density inversion machine learning model are used for soil bulk density prediction of the target farmland tillage layer.

### BRIEF DESCRIPTION OF THE DRAWINGS

FIG. 1 is a diagram showing training and actual measurement flows of the method;

FIG. 2 is a structural diagram of the system.

### DETAILED DESCRIPTION OF THE EMBODIMENTS

The specific implementations of the present disclosure are described below to facilitate understanding of the present disclosure by those skilled in the art. However, it should be clear that the present disclosure is not limited to the scope of the specific implementations. For those of ordinary skill in the art, as long as various variations fall within the spirit and scope of the present disclosure defined and determined by the appended claims, these variations are apparent, and all inventions and creations conceived by utilizing the concept of the present disclosure shall be within the scope of protection.

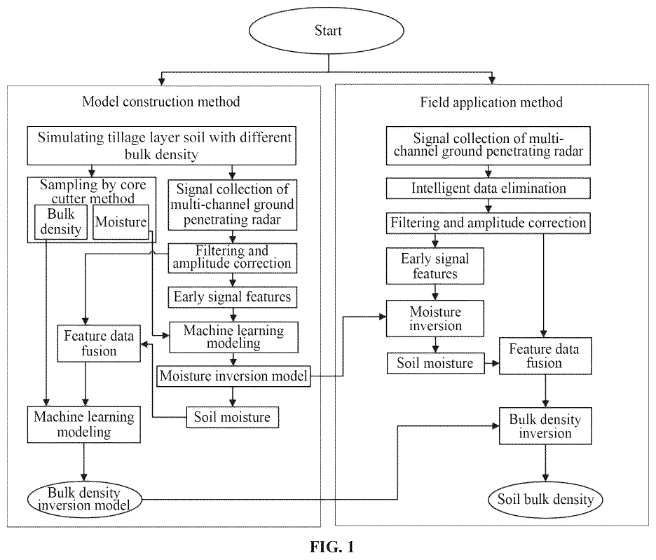

As shown in FIG. 1, a method for measuring soil bulk density of a farmland tillage layer based on a multi-channel ground penetrating radar is provided includes steps S1-S6.

In step S1, a relationship model (that is, a moisture inversion model) is constructed between the reciprocal of the average amplitude envelope of shallow electromagnetic signals acquired by antennas at different spacings and the volumetric soil water content in the farmland tillage layer.

In step S2, a trained bulk density inversion machine learning model (that is, a bulk density inversion model) is constructed.

In step S3, the target farmland tillage layer is detected through the ground penetrating radar to obtain an initial detection signal.

In step S4, the initial detection signal is preprocessed, and the reciprocal of the average amplitude envelope of the preprocessed signal is extracted within the time interval from time zero to a moment when the amplitude envelope reaches its maximum.

In step S5, based on the reciprocal of the average amplitude envelope obtained in step S4, the corresponding predicted volumetric water content is acquired according to the relationship model between the reciprocal of the average amplitude envelope and the volumetric soil water content in the farmland tillage layer.

In step S6, the reciprocal of the average amplitude envelope obtained in step S4 and the predicted volumetric water content obtained in step S5 are used as inputs of the trained bulk density inversion machine learning model, and soil bulk density of the farmland tillage layer is predicted through the trained bulk density inversion machine learning model to obtain a soil bulk density measurement result of the farmland tillage layer.

A specific method for constructing the relationship model between the reciprocal of the average amplitude envelope and the volumetric soil water content in the farmland tillage layer via testing in step S1 includes steps S1-1 to S1-6.

In step S1-1, soil from the target farmland tillage layer (within a depth of 20 cm) is selected and subjected to air-drying and sieving treatment under laboratory conditions.

In step S1-2, based on the soil treated in step S1-1, multiple simulated tillage layer samples are prepared by establishing several combinations (more than 5 combinations per case) of different bulk density and volumetric water content conditions, and these combinations are approximately evenly distributed across the suitable volumetric water content range and the bulk density measurement range (1.0 g/cm³-1.5 g/cm³). The corresponding simulated samples of tillage layer soil are uniformly packed into in a wooden box for testing, where a thickness of the soil in the wooden box is greater than or equal to 20 cm.

In step S1-3, the ground penetrating radar (with a central frequency from 400 MHz to 900 MHz) is placed on a surface of each simulated sample constructed in step S1-2 with more than three antenna spacings to collect an initial detection signal corresponding to each simulated sample.

In step S1-4, the initial detection signal corresponding to each simulated sample collected in step S1-3 is preprocessed, and the reciprocal of the average amplitude envelope of the preprocessed detection signal corresponding to each simulated sample is extracted within the time interval from time zero to the moment when the amplitude envelope reaches its maximum.

In step S1-5, each simulated sample is collected by using the core cutter method and dried, and the actual volumetric water content and bulk density of each simulated sample are then calculated, where a calculation formula of a water content θₓ after drying is as follows:

$${θ}_{X} = \frac{{W}_{w}}{{W}_{d}} \times 100 \% ,$$

where W_{w} is the mass of water (obtained by subtracting the mass of a dry soil sample from the mass of a wet soil sample), and W_{d} is the mass of the dry soil sample.

A calculation formula of the bulk density after drying is as follows:

$${ρ}_{X} = \frac{{W}_{d}}{V} ,$$

where V is the volume of the soil sample (determined by the volume of the cutting ring).

In step S1-6, a shallow neural network model is trained using the reciprocal of the average amplitude envelope obtained in step S1-4 as a training sample of the shallow neural network model and the actual volumetric water content of the simulated sample as a label of the training sample of the shallow neural network model to obtain the trained shallow neural network model, that is, the relationship model between the reciprocal of the average amplitude envelope and the volumetric soil water content in the farmland tillage layer is obtained.

In the process of training the shallow neural network model, stability of the shallow neural network model is evaluated through cross-validation, a prediction accuracy of the shallow neural network model is evaluated through a root mean square error metric, and the training of the shallow neural network model is finished when the stability and prediction accuracy of the shallow neural network model meet a first predetermined condition.

In this embodiment, the shallow neural network model uses a Back-propagation (BP) neural network including three hidden layers (with 20, 20 and 30 node neurons, respectively), Rectified Linear Unit (ReLu) is adopted as an activation function, the number of training epochs is 1000, and an initial learning rate is set to 10⁻³. Cross-validation (10 fold) is used to evaluate model stability, and the root mean square error (RMSE) metric is used to evaluate the model prediction accuracy. During normalization, assuming that a certain feature sample set of an original signal is Fᵢ (i=1, 2, . . . ) with a maximum value and a minimum value of Fₘₐₓ and Fₘᵢₙ, then a normalized value of the feature is as follows:

$${\hat{F}}_{i} = \frac{{F}_{i} - {F}_{min}}{{F}_{max} - {F}_{min}} .$$

A root mean square error calculation formula is as follows:

$$R M S E = \sqrt{\frac{1}{n} {\sum }_{i = 1}^{n} {( {O}_{i} - {P}_{i} )}^{2}} ,$$

where n represents the total number of the training samples, Oᵢ is a real value of an i^{-th} sample, and Pᵢ is a model predicted value of the i-th sample.

A specific method for constructing the trained bulk density inversion machine learning model in step S2 is implemented as follows:
a convolutional neural network model is trained using the corresponding reciprocal of the average amplitude envelope and the actual volumetric water content of each simulated sample as training samples and the actual bulk density of each simulated sample as a label to obtain a trained convolutional neural network model, that is, the trained bulk density inversion machine learning model is obtained.

In the process of training the convolutional neural network model, stability of the convolutional neural network model is evaluated through cross-validation (10 fold), a prediction accuracy of the convolutional neural network model is evaluated through a root mean square metric, and the training of the convolutional neural network model is finished when the stability and prediction accuracy of the convolutional neural network model meet a second predetermined condition.

In this embodiment, when the ground penetrating radar is equipped with four antennas, the initial detection signals correspond to four channels, yielding four reciprocals of the average amplitude envelopes. In this case, the reciprocals of the average amplitude envelopes and the predicted volumetric water content, when used as the inputs of the (trained) bulk density inversion machine learning model, can be expressed as: X=[A₁, A₂, A₃, A₄, W] where A₁, A₂, A₃, A₄ denote the reciprocals of the average amplitude envelopes of the four channels (representing the amplitude time-domain signal characteristics of four channels), respectively, and W is the predicted volumetric water content.

In specific implementation, the convolutional neural network model includes one convolutional layer (with 32 filters and a one-dimensional convolutional kernel of size 1*32) and three fully-connected layers (with the size of 128, 128, 128, respectively) The convolutional neural network mode uses ReLu as an activation function, is trained for 1000 epochs, and is configured with an initial learning rate of 10⁻⁵.

A specific method for preprocessing the initial detection signal is as follows:
performing time-zero calibration on the initial detection signal by using timing of WARR signals measured at different antenna spacings in air to obtain a time-zero-calibrated signal;
eliminating a low-frequency noise from the time-zero-calibrated signal by a De-Wow filter to obtain a low-frequency-filtered signal;
for the low-frequency-filtered signal, comparing amplitude energy differences among multiple sets of fixed-spacing airwaves from N measurements, setting the amplitude energy of fixed-spacing airwave from one selected measurement as a reference, and correcting the amplitude energies of the fixed-spacing airwaves from the remaining N−1 measurements to the same level to obtain a amplitude-corrected signal, that is, the preprocessed initial detection signal is obtained.

In this embodiment, airwave correction data from the first measurement can be directly set as a reference matrix, and a maximum average absolute value of an amplitude of each segment of signals across N channels and K spacings is calculated as reference energy Amax, where the reference matrix is constructed as follows:

$$Base = \{ \begin{matrix}A {max}_{1}^{1} & A {max}_{2}^{1} & … & A {max}_{K}^{1} \\ A {max}_{1}^{2} & A {max}_{2}^{2} & … & A {max}_{K}^{2} \\ … & … & … & … \\ A {max}_{1}^{N} & A {max}_{2}^{N} & … & A {max}_{K}^{N}\end{matrix} \} .$$

The airwave correction data of each test is calculated by using the foregoing method to obtain an amplitude energy matrix Obs, and then the amplitude energy matrix Obs is compared with the reference matrix Base to obtain a correction coefficient matrix Coef. A correction coefficient of each channel can be obtained by averaging correction coefficient matrix rows, and then the corrected amplitude is calculated, with the formula as follows:

$$\{ \begin{matrix}{Coef}_{i}^{*} = \frac{1}{K} {\sum }_{j = 1}^{K} {[ \frac{Obs}{Base} ]}_{ij} \\ {A}_{adjust , i} = {A}_{i} \cdot {Coef}_{i}^{*}\end{matrix} ,$$

where i is a channel number, j is an antenna spacing serial number, Aᵢ is an original amplitude of the i^{-th} channel, and A_{adjust,i} is a corrected amplitude of the i^{-th} channel.

When processing the preprocessed initial detection signal, the amplitude envelope E(t) of a time domain signal A(t) needs to be solved. The time domain signal A(t) is subjected to Hilbert transform:

$$\{ \begin{matrix}H ( t ) = A ( t ) \times h ( t ) \\ h ( t ) = \frac{1}{π t}\end{matrix} ;$$

where h(t) is a kernel function of Hilbert transform, which is used for the convolutional operation, H(t) is the Hilbert transform of A(t). And then the amplitude envelope is obtained as follows:

$$E ( t ) = \sqrt{{A}^{2} ( t ) + {H}^{2} ( t )} .$$

Then, the reciprocal of the average amplitude envelope (AEA⁻¹) can be extracted for each channel within the time interval from time zero to the moment when the amplitude envelope reaches its maximum.

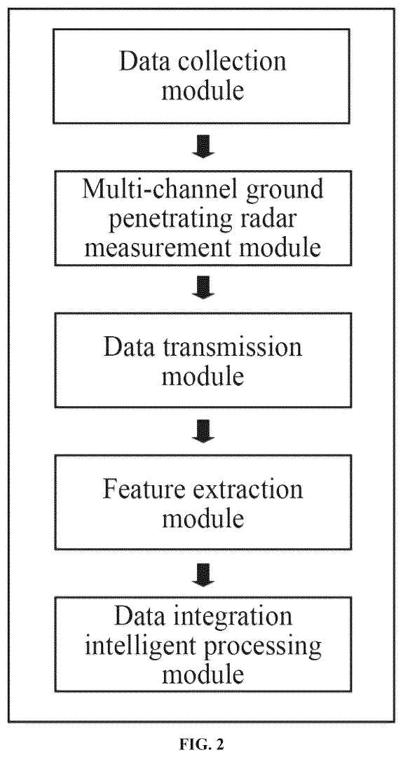

As shown in FIG. 2, a system for implementing a method for measuring soil bulk density of a farmland tillage layer based on a multi-channel ground penetrating radar includes a data collection module, a multi-channel ground penetrating radar measurement module, a data transmission module, and an upper computer.

The data collection module includes a camera and a Real-Time Kinematic (RTK) positioning device, where the camera is configured to collect a surface image of a target farmland tillage layer, and the RTK positioning device is configured to monitor position information of a point detected by a ground penetrating radar in real time via RTK positioning technology.

The multi-channel ground penetrating radar measurement module is configured to detect the target farmland tillage layer to acquire an initial detection signal.

The data transmission module is configured to transmit data obtained by the data collection module and the multi-channel ground penetrating radar measurement module to the upper computer via a wireless communication network.

The upper computer includes a feature extraction module and a data integration intelligent processing module. The feature extraction module is configured to preprocess the initial detection signal and extract a reciprocal of an average amplitude envelope of the preprocessed initial detection signal within a time interval from time zero to the moment when the amplitude envelope reaches its maximum.

The data integration intelligent processing module is configured to: construct a relationship model between the reciprocal of the average amplitude envelope and a volumetric soil water content in the farmland tillage layer, construct a trained bulk density inversion machine learning model, acquire a corresponding predicted volumetric water content based on the relationship model between the reciprocal of the average amplitude envelope and the volumetric soil water content in the farmland tillage layer, predict, based on the reciprocal of the average amplitude envelope and the predicted volumetric soil water content, soil bulk density of the farmland tillage layer through the trained bulk density inversion machine learning model to obtain a soil bulk density measurement result of the farmland tillage layer, and store and display data collected by the data collection module with corresponding soil bulk density measurement result of the farmland tillage layer.

In an embodiment of the present disclosure, when measuring the soil bulk density of the farmland tillage layer in the field, the parameter settings and operation procedures of the ground-penetrating radar should be consistent with those used in a test for constructing the trained bulk density inversion machine learning model. The data integration intelligent processing module can be configured to identify and filter out abnormal points such as those caused by uneven surface and rutting according to surface images and topographic variation information acquired by the data collection module.

In conclusion, through the collection of the ground penetrating radar and other detection data, multiple data features are fused by using multiple data processing methods, and the soil bulk density of the large-area tillage layer is measured by combining with a machine learning algorithm. The efficiency and accuracy of measurement can be improved, and the problem that the existing soil bulk density detection method is difficult to achieve large-area, rapid and continuous detection and unsuitable for measuring the loose tillage layer soil is solved.

What is claimed is:

1. A method for measuring soil bulk density of a farmland tillage layer based on a multi-channel ground penetrating radar, comprising following steps:
step S1: constructing a relationship model between a reciprocal of an average amplitude envelope and a volumetric soil water content in a farmland tillage layer;
step S2: constructing a trained bulk density inversion machine learning model;
step S3: obtaining an initial detection signal by detecting a target farmland tillage layer through a ground penetrating radar;
step S4: preprocessing the initial detection signal, and extracting a reciprocal of an average amplitude envelope of preprocessed initial detection signal within a time interval from time zero to a moment when an amplitude envelope reaches maximum;
step S5: obtaining a corresponding predicted volumetric water content according to the relationship model between the reciprocal of the average amplitude envelope and the volumetric soil water content in the farmland tillage layer based on the reciprocal of the average amplitude envelope obtained in step S4; and
step S6: serving the reciprocal of the average amplitude envelope of a tillage layer electromagnetic wave signal acquired by antennas with different spacings obtained in step S4 and the predicted volumetric water content obtained in step S5 as inputs of the trained bulk density inversion machine learning model, and predicting soil bulk density of the farmland tillage layer through the trained bulk density inversion machine learning model to obtain a soil bulk density measurement result of the farmland tillage layer;
a specific method for constructing the trained bulk density inversion machine learning model in step S2 comprises:
training a convolutional neural network model with a corresponding reciprocal of an average amplitude envelope and an actual volumetric water content of a simulated sample as a training sample of the convolutional neural network and an actual bulk density of the simulated sample as a label to obtain a trained convolutional neural network model, that is, obtaining the trained bulk density inversion machine learning model.

2. The method for measuring the soil bulk density of the farmland tillage layer based on the multi-channel ground penetrating radar according to claim 1, wherein a specific method for constructing the relationship model between the reciprocal of the average amplitude envelope and the volumetric soil water content in the farmland tillage layer through testing in step S1 comprises following sub-steps:
step S1-1: selecting soil from the target farmland tillage layer for air-drying and sieving treatment;
step S1-2: based on the soil treated in step S1-1, constructing a plurality of simulated samples with different bulk densities and volumetric water content conditions in a wooden box, wherein a soil thickness in the wooden box is greater than or equal to 20 cm;
step S1-3: placing the ground penetrating radar on a surface of each simulated sample constructed in step S1-2 with more than three antenna spacings to collect an initial detection signal corresponding to each simulated sample;
step S1-4: preprocessing the initial detection signal corresponding to each simulated sample collected in step S1-3, and extracting a reciprocal of an average amplitude envelope of a preprocessed initial detection signal corresponding to each simulated sample within a time interval from time zero to a moment when an amplitude envelope reaches maximum;
step S1-5: sampling each simulated sample by using a core cutter method and drying each simulated sample, and calculating an actual volumetric water content and bulk density of each simulated sample; and
step S1-6: training a shallow neural network model by using the reciprocal of the average amplitude envelope obtained in step S1-4 as a training sample of the shallow neural network model and the actual volumetric water content of each simulated sample as a label of the shallow neural network model to obtain a trained shallow neural network model, that is, obtaining the relationship model between the reciprocal of the average amplitude envelope and the volumetric soil water content in the farmland tillage layer.

3. The method for the measuring soil bulk density of the farmland tillage layer based on the multi-channel ground penetrating radar according to claim 2, wherein in a process of training the shallow neural network model, stability of the shallow neural network model is evaluated through cross-validation, a prediction accuracy of the shallow neural network model is evaluated through a root mean square error metric, and a training of the shallow neural network model is finished when the stability and the prediction accuracy of the shallow neural network model meet a predetermined condition.

4. The method for measuring the soil bulk density of the farmland tillage layer based on the multi-channel ground penetrating radar according to claim 1, wherein in a process of training the convolutional neural network model, stability of the convolutional neural network model is evaluated through cross-validation, a prediction accuracy of the convolutional neural network model is evaluated through a root mean square error metric, and a training of the convolutional neural network model is finished when the stability and the prediction accuracy of the convolutional neural network model meet a predetermined condition.

5. The method for measuring the soil bulk density of the farmland tillage layer based on the multi-channel ground penetrating radar according to claim 1, wherein a specific method for preprocessing the initial detection signal comprises:
performing time-zero calibration on the initial detection signal by using timing of Wide Angle Reflection and Refraction (WARR) signals measured at different spacings in air to obtain a time-zero calibrated signal;
eliminating a low-frequency noise from the time-zero calibrated signal by a De-Wow filter to obtain a low-frequency filtered signal; and
for the low-frequency filtered signal, comparing amplitude energy differences among multiple sets of fixed-spacing airwaves from N measurements, setting an amplitude energy of fixed-spacing airwave from one selected measurement as a reference, and correcting amplitude energies of fixed-spacing airwaves from remaining N−1 measurements to a same level to obtain an amplitude corrected signal, that is, obtaining the preprocessed initial detection signal.

6. A system for implementing the method for measuring the soil bulk density of the farmland tillage layer based on the multi-channel ground penetrating radar according to claim 1, comprising a data collection module, a multi-channel ground penetrating radar measurement module, a data transmission module, and an upper computer; wherein
the data collection module comprises a camera and a Real-Time Kinematic (RTK) positioning device, wherein the camera is configured to collect a surface image of a target farmland tillage layer, and the RTK positioning device is configured to monitor position information of a point detected by the ground penetrating radar in real time by means of RTK positioning technology;
the multi-channel ground penetrating radar measurement module is configured to detect the target farmland tillage layer to acquire an initial detection signal;
the data transmission module is configured to transmit data obtained by the data collection module and the multi-channel ground penetrating radar measurement module to the upper computer through a wireless communication network; and
the upper computer comprises a feature extraction module and a data integration intelligent processing module, wherein the feature extraction module is configured to preprocess the initial detection signal and extract the reciprocal of the average amplitude envelope of the preprocessed initial detection signal within the time interval from time zero to the moment when the amplitude envelope reaches maximum; and
the data integration intelligent processing module is configured to: construct the relationship model between the reciprocal of the average amplitude envelope and the volumetric soil water content in the farmland tillage layer; construct the trained bulk density inversion machine learning model; acquire the corresponding predicted volumetric water content according to the relationship model between the reciprocal of the average amplitude envelope and the volumetric soil water content in the farmland tillage layer; predict, based on the reciprocal of the average amplitude envelope and the predicted volumetric soil water content, the soil bulk density of the farmland tillage layer through the trained bulk density inversion machine learning model to obtain the soil bulk density measurement result of the farmland tillage layer; and store and display data collected by the data collection module with corresponding soil bulk density measurement result of the farmland tillage layer.

7. The system for implementing the method for measuring the soil bulk density of the farmland tillage layer based on the multi-channel ground penetrating radar according to claim 6, wherein a specific method for constructing the relationship model between the reciprocal of the average amplitude envelope and the volumetric soil water content in the farmland tillage layer through testing in step S1 comprises following sub-steps:
step S1-1: selecting soil from the target farmland tillage layer for air-drying and sieving treatment;
step S1-2: based on the soil treated in step S1-1, constructing a plurality of simulated samples with different bulk densities and volumetric water content conditions in a wooden box, wherein a soil thickness in the wooden box is greater than or equal to 20 cm;
step S1-3: placing the ground penetrating radar on a surface of each simulated sample constructed in step S1-2 with more than three antenna spacings to collect an initial detection signal corresponding to each simulated sample;
step S1-4: preprocessing the initial detection signal corresponding to each simulated sample collected in step S1-3, and extracting a reciprocal of an average amplitude envelope of a preprocessed initial detection signal corresponding to each simulated sample within a time interval from time zero to a moment when an amplitude envelope reaches maximum;
step S1-5: sampling each simulated sample by using a core cutter method and drying each simulated sample, and calculating an actual volumetric water content and bulk density of each simulated sample; and
step S1-6: training a shallow neural network model by using the reciprocal of the average amplitude envelope obtained in step S1-4 as a training sample of the shallow neural network model and the actual volumetric water content of each simulated sample as a label of the shallow neural network model to obtain a trained shallow neural network model, that is, obtaining the relationship model between the reciprocal of the average amplitude envelope and the volumetric soil water content in the farmland tillage layer.

8. The system for implementing the method for measuring the soil bulk density of the farmland tillage layer based on the multi-channel ground penetrating radar according to claim 7, wherein in a process of training the shallow neural network model, stability of the shallow neural network model is evaluated through cross-validation, a prediction accuracy of the shallow neural network model is evaluated through a root mean square error metric, and a training of the shallow neural network model is finished when the stability and the prediction accuracy of the shallow neural network model meet a predetermined condition.

9. The system for implementing the method for measuring the soil bulk density of the farmland tillage layer based on the multi-channel ground penetrating radar according to claim 6, wherein in a process of training the convolutional neural network model, stability of the convolutional neural network model is evaluated through cross-validation, a prediction accuracy of the convolutional neural network model is evaluated through a root mean square error metric, and a training of the convolutional neural network model is finished when the stability and the prediction accuracy of the convolutional neural network model meet a predetermined condition.

10. The system for implementing the method for measuring the soil bulk density of the farmland tillage layer based on the multi-channel ground penetrating radar according to claim 6, wherein a specific method for preprocessing the initial detection signal comprises:
performing time-zero calibration on the initial detection signal by using timing of Wide Angle Reflection and Refraction (WARR) signals measured at different spacings in air to obtain a time-zero calibrated signal;
eliminating a low-frequency noise from the time-zero calibrated signal by a De-Wow filter to obtain a low-frequency filtered signal; and
for the low-frequency filtered signal, comparing amplitude energy differences among multiple sets of fixed-spacing airwaves from N measurements, setting an amplitude energy of fixed-spacing airwave from one selected measurement as a reference, and correcting amplitude energies of fixed-spacing airwaves from remaining N−1 measurements to a same level to obtain an amplitude corrected signal, that is, obtaining the preprocessed initial detection signal.
